(19) United States
(12) Patent Application Publication
(10) Pub. No.: US 2014/0228968 A1
(43) Pub. Date: Aug. 14, 2014

(54) BIOCORRODIBLE IMPLANT WITH ANTI-CORROSION COATING

(71) Applicant: BIOTRONIK AG, Buelach (CH)

(72) Inventor: Eric Wittchow, Gunzenhausen (DE)

(73) Assignee: BIOTRONIK AG, Buelach (CH)

(21) Appl. No.: 14/166,419

(22) Filed: Jan. 28, 2014

Related U.S. Application Data
(60) Provisional application No. 61/763,955, filed on Feb. 13, 2013.

(51) Int. Cl. A61L 31/14; A61L 31/10; A61F 2/82

(52) U.S. Cl. CPC A61L 31/148; A61F 2/82; A61L 31/10
USPC 623/23.7

(57) ABSTRACT

An implant comprising a main body made of magnesium or a biocorrodible magnesium alloy and a corrosion-inhibiting passivation layer covering the main body. The passivation layer is characterized in that it contains a 3-polyhydroxyalkanoate and the 3-polyhydroxyalkanoate is a homomer or copolymer with a polymer segment of formula (1):

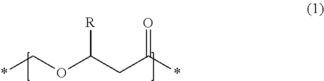

where R=propyl, butyl, pentyl or hexyl.

### CROSS REFERENCE TO RELATED APPLICATIONS

This invention claims benefit of priority to U.S. provisional patent application no. 61/763,955 filed Feb. 13, 2013; the content of which is herein incorporated by reference in its entirety.

### TECHNICAL FIELD

The invention relates to a biocorrodible implant based on magnesium or a biocorrodible magnesium alloy, of which the surface has an anti-corrosion coating.

### BACKGROUND

An implant is generally understood to mean any medical device formed from one or more materials, which is inserted intentionally into the body and is covered either in part or completely by an epithelial surface. Implants can be sub-divided in terms of the period of use into temporary and permanent implants. Temporary implants remain in the body for a limited period of time. Permanent implants are intended to remain permanently in the body. In the case of implants, a further distinction can be made between prostheses and artificial organs. A prosthesis is a medical device that replaces limbs, organs or tissues of the body, whereas an artificial organ is understood to be a medical device that replaces the function of a bodily organ, either in part or in full. The above definitions include, for example, implants such as orthopedic or osteosynthetic implants, cardiac pacemakers and defibrillators and vascular implants.

In particular, the implantation of stents has become established as one of the most effective therapeutic measures in the treatment of vascular diseases. Stents are used to perform a supporting function in a patient's hollow organs. For this purpose, stents of conventional design have a filigree supporting structure formed from metal struts, which is initially provided in a compressed form for insertion into the body and is expanded at the site of application. One of the main fields of application of such stents is the permanent or temporary widening and maintained opening of vascular constrictions, in particular of constrictions (stenoses) of the coronary vessels. In addition, aneurysm stents are known for example, which are used primarily to seal the aneurysm.

Stents have a peripheral wall of sufficient supporting strength to hold open the constricted vessel to the desired extent and also a tubular main body, through which the flow of blood continues unimpeded. The peripheral wall is generally formed by a mesh-like supporting structure, which allows the stent to be introduced in a compressed state with small outer o diameter as far as the narrowed point to be treated of the respective vessel, where said stent can then be expanded with the aid of a balloon catheter for example until the vessel has the desired, increased inner diameter. Alternatively, shape-memory materials such as Nitinol have the ability to self-expand if there is no restoring force that holds the implant at a small diameter. The restoring force is generally exerted onto the material by means of a protective tube.

The implant, in particular the stent, has a main body formed from an implant material. An implant material is a non-living material, which is used for an application in the field of medicine and interacts with biological systems. Basic preconditions for the use of a material as implant material that comes into contact with the bodily environment when used as intended is its compatibility with the body (biocompatibility). Biocompatibility is understood to mean the ability of a material to induce a suitable tissue response in a specific application. This includes an adaptation of the chemical, physical, biological and morphological surface properties of an implant to the receiver tissue with the objective of a clinically desired interaction. The biocompatibility of the implant material is also dependent on the progression over time of the response of the biosystem into which the material has been implanted. Relatively short-term irritation and inflammation thus occur, which may lead to tissue changes. Biological systems therefore respond differently according to the properties of the implant material. The implant materials can be divided into bioactive, bioinert and degradable/resorbable materials in accordance with the response of the biosystem.

Implant materials comprise polymers, metal materials and ceramic materials (for example as a coating). Biocompatible metals and metal alloys for permanent implants include stainless steels for example (such as 316L), cobalt-based alloys (such as CoCrMo cast alloys, CoCrMo forged alloys, CoCrWNi forged alloys and CoCrNiMo forged alloys), pure titanium and titanium alloys (for example cp titanium, TiAl6V4 or TiAl6Nb7) and gold alloys. In the field of biocorrodible implants, in particular stents, the use of magnesium or pure iron as well as biocorrodible master alloys of the elements magnesium, iron, zinc, molybdenum and tungsten is recommended.

For the purposes of the present invention, merely metal implant materials that consist completely or in part of magnesium or biocorrodible magnesium alloys are of interest.

A problem with the use of biocorrodible magnesium alloys is the rapid degradation of the material in a physiological environment. Both the fundamental principles of magnesium corrosion and a large number of technical methods for improving the corrosion behavior (in the sense of boosting the protection against corrosion) are known from the prior art. For example, it is known that the addition of yttrium and/or further rare earth metals of a magnesium alloy provides slightly increased resistance to corrosion in seawater.

A starting point for improving corrosion behavior lies in producing an anti-corrosion layer on the shaped article consisting of magnesium or a magnesium alloy. Known methods for producing an anti-corrosion layer have been previously developed and optimized from the viewpoint of a technical use of the shaped article, but not from the viewpoint of a medical use in biocorrodible implants in a physiological environment. These known methods comprise: the application of polymers or inorganic cover layers, the production of an enamel, the chemical conversion of the surface, hot-gas oxidation, anodizing, plasma spraying, laser beam remelting, PVD methods, ion implantation or coating.

Conventional technical fields of use of shaped articles made of magnesium alloys outside the field of medicine generally require extensive prevention of corrosive processes. The objective of most technical methods is accordingly complete elimination of corrosive processes. By contrast, the objective for improving the corrosive behavior of biocorrodible magnesium alloys should not lie in complete prevention, but merely in the suppression of corrosive processes. For this reason alone, most known methods for the production of anti-corrosion layers on magnesium are unsuitable. Furthermore, toxicological aspects also have to be taken into account for medical use. Corrosive processes are also highly dependent on the medium in which they take place, and it may not therefore be possible to transfer, without limitation, the anti-corrosion findings obtained under conventional environmental conditions in the technical field to the processes in a physiological environment. Lastly, with a large number of medical implants the mechanisms forming the basis of corrosion may also differ from conventional technical applications of the material. For example, stents, surgical material or clips are mechanically deformed during use, and therefore the sub-process of stress corrosion cracking could be of considerable importance during the breakdown of these shaped articles.

The main body of some implants, such as stents in particular, is subject locally during use to plastic deformation of varying strength. Conventional methods for inhibiting corrosion, such as the generation of a tight magnesium oxide cover layer, do not lead to the desired result in this instance. The ceramic properties of such a cover layer would lead to local flaking of the cover layer. The corrosion would therefore occur in an uncontrolled manner and in particular there would be a risk that the corrosion is accelerated in the regions of the implant subject to considerable mechanical stress.

The application of non-degradable, polymer passivation layers can indeed inhibit the breakdown of the implant, but this contradicts the fundamental idea of a fully degradable implant, since the polymer material of the passivation layer remains in the body. A promising alternative is therefore the use of biodegradable polymers as material for a passivation layer for implants based on biodegradable magnesium materials.

It is known from US 2009/0240323 A1, US 2010/0076544 A1 and US 2011/0076319 A1 to coat a magnesium stent with a biodegradable polymer coating as a passivation layer. The breakdown of the main body made of magnesium or a magnesium alloy should therefore be decelerated. For example, poly(lactides), polyhydroxyalkanoates (in particular polyhydroxyvalerate), polycaprolcatones, aliphatic polyesters, aromatic copolyesters and polyesteramides are possible biodegradable polymer coating materials. In spite of these promising approaches, it has not yet been possible to satisfactorily delay to a sufficient degree the degradation of the metal main body of the stent and to simultaneously meet all further requirements of the coating, in particular such as sufficient elongation at failure.

### SUMMARY OF THE INVENTION

One or more of the discussed problems of the prior art can be overcome or at least mitigated with the aid of the implant according to the invention. The implant, preferably a stent, has a main body made of magnesium or a biocorrodible magnesium alloy and a corrosion-inhibiting passivation layer covering the main body. The passivation layer is characterized in that it contains a 3-polyhydroxyalkanoate and the 3-polyhydroxyalkanoate is a homomer or copolymer with a polymer segment of formula (1):

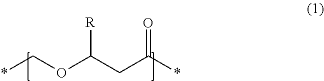

where R=propyl, butyl, pentyl or hexyl. The 3-polyhydroxyalkanoate is preferably selected from the group comprising polyhydroxyhexanoate (PHHx), polyhydroxyheptanoate (PHHp), polyhydroxyoctonoate (PHO) and polyhydroxynonanoate (PHN).

### DETAILED DESCRIPTION OF PREFERRED EMBODIMENTS

The invention includes an implant, preferably a stent, having a main body made of magnesium or a biocorrodible magnesium alloy and a corrosion-inhibiting passivation layer covering the main body. The passivation layer is characterized in that it contains a 3-polyhydroxyalkanoate and the 3-polyhydroxyalkanoate is a homomer or copolymer with a polymer segment of formula (1):

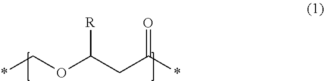

where R=propyl, butyl, pentyl or hexyl. The 3-polyhydroxyalkanoate is preferably selected from the group comprising polyhydroxyhexanoate (PHHx), polyhydroxyheptanoate (PHHp), polyhydroxyoctonoate (PHO) and polyhydroxynonanoate (PHN).

Polyhydroxyalkanoates (PHAs) are naturally occurring water-insoluble and linear polyesters. The polymers are biologically degradable and are used for the production of plastics, but also as biodegradable material for medical products, such as suture materials, for implants and for pharmaceutical depot preparations. Depending on the microorganism and cultivation conditions used, the biosynthesis of PHAs delivers various homo- or copolyesters, which differ in terms of their properties. The polymers are generally UV-stable, withstand processing temperatures of up to 180° C. and demonstrate slight permeation of water. The simplest and most frequently occurring form of PHAs is the fermentatively produced poly(R-3-hydroxybutyrate) (polyhydroxybutyric acid, PHB or poly(3HB). PHB and further short-chain PHAs are relatively brittle and stiff however.

The invention is based on the finding that the use of long-chain polyhydroxyalkanoates with polyhydroxy fatty acids derived from C6 to C9 as coating materials for implants, (in particular for stents) made of magnesium or magnesium alloys, provides specific advantages. On the one hand, the impermeability to water of the passivation layer is very low, which leads to temporary inhibition of the corrosion of the underlying metal main body. On the other hand, the material demonstrates behavior that is particularly advantageous for the intended purpose, in particular the elongation at failure of the polymer material is sufficiently high. In the event of expansion of stents, microcracks for example, which could cause inhomogeneous breakdown behavior of the implant, therefore do not form in the passivation layer.

The passivation layer may contain further additives and preferably also an active ingredient, which are released after implantation.

Exemplary embodiment—coating of an absorbable metal stent made of a magnesium alloy with a polyhydroxyhexanoate (PHHx) charged with active ingredient.

The stent is cleaned of dust and residues and is fixed in a suitable stent-coating machine (DES Coater, own development by Biotronik). For the coating, a solution of 0.1% by weight of PHHx and 0.05% by weight of a pharmacological active ingredient (for example rapamycin) in chloroform is prepared. With the aid of an airbrush system the rotating stent is coated on one side under constant ambient conditions (room temperature; 42% rH). With a nozzle distance of 20 mm, a stent 18 mm long is coated after approximately 10 min. Once the predefined layer mass has been reached, the stent is dried for 5 min at room temperature and the stent is then rotated and fixed again, and the uncoated side is coated in the same way. The finished, coated stent is dried for 24 h at 80° C. in a vacuum oven.

It will be apparent to those skilled in the art that numerous modifications and variations of the described examples and embodiments are possible in light of the above teaching. The disclosed examples and embodiments are presented for purposes of illustration only. Other alternate embodiments may include some or all of the features disclosed herein. Therefore, it is the intent to cover all such modifications and alternate embodiments as may come within the true scope of this invention.

What is claimed is:

1. An implant comprising a main body made of magnesium or a biocorrodible magnesium alloy and a corrosion-inhibiting passivation layer covering the main body, wherein the passivation layer contains a 3-polyhydroxyalkanoate and the 3-polyhydroxyalkanoate is a homomer or copolymer with a polymer segment of formula: 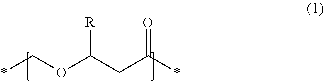
where R=propyl, butyl, pentyl or hexyl.

2. The implant as claimed in claim 1, wherein the 3-polyhydroxyalkanoate is selected from the group consisting of polyhydroxyhexanoate (PHHx), polyhydroxyheptanoate (PHHp), polyhydroxyoctonoate (PHO), and polyhydroxynonanoate (PHN).

3. The implant as claimed in claim 1, wherein the implant is a stent.
